(19) United States
(12) Patent Application Publication
(10) Pub. No.: US 2016/0166621 A1
(43) Pub. Date: Jun. 16, 2016

(54) NON-CHEMOTHERAPEUTIC ANTIBIOTIC TREATMENT FOR INFECTIONS IN CATTLE

(71) Applicants: THE BOARD OF REGENTS FOR OKLAHOMA STATE UNIVERSITY, STILLWATER, OK (US); THE BOARD OF REGENTS OF THE UNIVERSITY OF OKLAHOMA, NORMAN, OK (US)

(72) Inventors: Melanie J. Boileau, Glencoe, OK (US); Kenneth D. Clinkenbeard, Stillwater, OK (US); Rinosh Joshua Mani, Stillwater, OK (US); John J. Iandolo, Edmond, OK (US); Huda Mussa, Oklahoma City, OK (US)

(21) Appl. No.: 15/047,906

(22) Filed: Feb. 19, 2016

Related U.S. Application Data
(62) Division of application No. 14/348,824, filed on Mar. 31, 2014.
(60) Provisional application No. 61/541,777, filed on Sep. 30, 2011.

(51) Int. Cl. A61K 35/74; A61K 9/00; A61K 9/19; C12N 1/20

(52) U.S. Cl. CPC A61K 35/74; C12N 1/20; A61K 9/0048; A61K 9/0073; A61K 9/19

(57) ABSTRACT

Compositions and methods for treating and/or preventing bacterial infections in animals such as bovines are provided. The compositions and methods utilize the predatory bacterium Bdellovibrio bacteriovorus to treat and/or prevent infections such as infectious bovine keratoconjunctivitis (IBK) and bovine respiratory disease (BRD). Use of the bacterium obviates the need for chemotherapeutic measures.

### STATEMENT REGARDING FEDERALLY SPONSORED RESEARCH OR DEVELOPMENT

This invention was made with United States government support under NIH Grant No. 5 P20 RR015564 awarded by the National Institutes of Health. The US government has certain rights in this invention.

### FIELD OF THE INVENTION

The invention generally relates to the treatment and/or prevention of infections in animals such as bovines. In particular, the invention provides compositions and methods which utilize the predatory bacterium Bdellovibrio bacteriovorus (e.g. B. bacteriovorus 109J) to treat and/or prevent infections such as infectious bovine keratoconjunctivitis (IBK) and bovine respiratory disease (BRD).

### BACKGROUND OF THE INVENTION

Ocular infection of cattle with Moraxella bovis is associated with the development of infectious bovine keratoconjunctivitis (IBK), known colloquially as “pink eye”. It is a widespread, severe, contagious eye disease of cattle that causes significant economic loss worldwide, including reduced productivity in beef (poor weight gain pre- and post-weaning) and dairy cattle industry (decreased milk production, milk discard in treated animal), direct cost of repeated antibiotic treatments, and devaluation (especially in show animals) due to eye disfigurement or blindness. IBK can be observed throughout the year, although outbreaks are more prevalent in the summer months when ultraviolet light, dust, wind and tall grass irritate the cornea (eye surface) and face flies are abundant, predisposing to the disease. Transmission occurs primarily via face flies which act as carriers of M. bovis, and occasionally through direct contact between infected and non-infected cattle or by indirect contact with contaminated fomites (e.g. a halter, stanchion, etc.). The clinical manifestations of IBK range from mild conjunctivitis (red eye) and excessive tearing to central corneal ulceration and perforation. If left untreated, affected animals may become blind due to severe blepharospasms (excessive squinting), corneal edema (hazy eye) or corneal perforation. Diagnosis of IBK is usually based on clinical signs and can be confirmed via culture of a swab from affected eye(s), with bacterial cultures collected from ocular secretions of calves with corneal ulcer due to M. bovis harboring 1×10⁹ to 1×10¹⁰ M. bovis per sample.

M. bovis fimbriae (Q pili) allow for bacterial adherence to the bovine conjunctival mucosa. Prerequisites for induction of ocular lesions by M. bovis include microbial adhesion to the corneal surface and cytotoxicity, both mediated by several virulence factors. The cytopathic effect of M. bovis on bovine corneal epithelial cells, neutrophils, and erythrocytes is mediated by a cytotoxic and leukotoxic hemolysin, hydrolytic and lipolytic enzymes, proteases, and collagenases. However, this damage caused by infection can be reversible as the corneal epithelium may regenerate once ocular M. bovis infection is cleared.

Administration of injectable antibiotics such as oxytetracycline (LA200®), florfenicol (Nuflor®), ceftiofur crystalline free acid (Excede®) and tulathromycin (Draxxin®) have been shown effective in the treatment of experimentally induced and naturally occurring IBK. However treatment failures are common and current commercially available vaccines are not optimally effective due mainly to M. bovis antigenic strain variation.

Bovine respiratory disease complex (BRD) is the most costly disease of beef cattle in North America. It is one of the most extensively studied diseases, with research that began with its description in the late 1800's. Mannheimia haemolytica, Pasteurella multocida, Histophilus somni (all Gram-negative bacteria) and Mycoplasma bovis have been clearly linked with BRD. Despite overwhelming evidence that these bacterial species are at least associated with, if not the principle cause of BRD, researchers have been unable to replicate the typical clinical presentation through experimental exposure to the bacterium alone. Therefore, BRD is a multi-factorial syndrome with some combination of predisposing factors being necessary to induce natural disease. Commonly cited factors include viral infections (infectious bovine rhinotracheitis, bovine viral diarrhea virus, parainfluenza 3, bovine respiratory syncytial virus, and bovine coronavirus) and environmental “stressors.” These latter include transportation, co-mingling, dust, weather extremes, dehydration, and acute metabolic disturbances. While it is known that M. haemolytica and P. multocida can be found in the upper respiratory tract of healthy cattle, it appears that heavy growth from the nasopharynx in cattle with clinical signs of BRD has some ability to predict what organism(s) is/are present in the lung.

Vaccination is the most widely practiced measure taken throughout the industry to protect against BRD. Vaccines exist in numerous forms and combinations for many viral agents and several bacteria implicated in BRD. Many studies have shown protection resulting from vaccinating with one or more products. However, a similar number of studies have found that vaccination was ineffective or inconclusive. While vaccination is consistently shown to result in antibody production, vaccination induced titers are not always correlated with protection against disease. Compared to vaccination, the practice of metaphylaxis (defined as administration of parenteral products to calves that are at high risk for BRD) has more consistently been shown to reduce clinical disease. Other products with consistent positive results in treating BRD include antibiotics such as florfenicol (Nuflor®), tilmicosin (Micotil®) and tulathromycin (Draxxin®). However, the use of antibiotics in cattle has become controversial, and even when successful, requires that milk produced by a treated lactating dairy cow be discarded. Furthermore, meat from treated beef cattle cannot enter the food chain until the proper withdrawal time as been followed. There is an ongoing need in the art to develop new strategies for the prevention and treatment of infection in livestock such as cattle, and especially to develop new strategies that do not involve the use of antibiotics or other chemotherapeutics.

### SUMMARY

The present invention provides methods and compositions for treating and/or preventing infections in livestock by using the predatory bacterium Bdellovibrio bacteriovorus (e.g. B. bacteriovorus 109J). The methods and compositions advantageously obviate the use of chemotherapeutics such as antibiotics. Exemplary infections which may be treated in this manner include but are not limited to IBK and BRD in cattle.

### BRIEF DESCRIPTION OF THE DRAWINGS

FIG. 1: Time to active B. bacteriovorus predatory morphology for passages 1 through 6 of M. bovis in comparison to normal prey E. coli.

FIG. 2: Efficacy of B. bacteriovorus predation of M. bovis in broth cultures. Initial inocula levels of M. bovis (CFU/mL) are plotted on the x-axis versus the B. bacteriovorus killing efficacy (%) at 4 h of exposure on the y-axis. Low and high passages of B. bacteriovorus on M. bovis correspond to 2 to 3 and 6 passages, respectively.

FIG. 3: Light microscopic appearance of M. bovis adhering to Madin-Darby bovine kidney (MDBK) cells. Moraxella bovis attached to MDBK cells appeared as darkly stained rods or coccobacillary shapes, characteristically in pairs, adhered to the surface of the larger polygonal MDBK cells. Wright-Giemsa stain. Magnification: 1000×. Bar=4.5 μm.

FIG. 4. Resurrection of B. bacteriovorus after lyophilization. GE: genomic equivalent; X1=B. bacteriovorus (log GE/mL) prior to lyophilization; X2=B. bacteriovorus (log GE/mL) following 24 hours of lyophilization (e.g. lyophilized powder+sterile water); T24 h=B. bacteriovorus inoculum (log GE/mL) (e.g. X2) diluted into standard liquid culture with non-hemolytic M. bovis (1:9 dilution). T48 h, T72 h and T96 h=B. bacteriovorus (log GE/mL) in standard liquid culture at 48, 72, and 96 hours, respectively. Overall loss of B. bacteriovorus following lyophilization is 1 to 1.6 log on average.

### DETAILED DESCRIPTION OF THE PREFERRED EMBODIMENTS

B. bacteriovorus is a small (0.35×1.2 μm), obligate aerobe, motile (polar flagellum), Gram-negative bacterium with obligate host-dependency on a wide range of Gram-negative prey bacteria. The genus Bdellovibrio was first described by Stolp and Starr in 1963 as bacteriolytic organisms capable of attacking a living bacterium, attaching to its surface, penetrating the cell wall, multiplying inside the host, and causing lysis of the infested cell (Stolp and Starr. Antonie Van Leeuwenhoek J Microbiol Serol 1963; 29:217-248). This occurs within approximately 3.5 to 4 hours. More recent reviews have described the predatory lifestyle of B. bacteriovorus as characterized by 2 differentiated cell stages (attack and growth phase). Methods for laboratory maintenance are also known. B. bacteriovorus are ubiquitous and have been isolated from terrestrial and aquatic environments including soils, rice paddies, rhizosphere of plants, rivers, sewage, fish ponds, and irrigation water. Despite the use of Escherichia coli as the model prey bacterium in the majority of in vitro experiments published, B. bacteriovorus is selectively active against most Pseudomonas spp. and enterobacteria. Although variable between prey cells, a minimum prey density is required to sustain the B. bacteriovorus life cycle. In 2 different studies, a prey concentration of approximately 1.5×10⁵ E. coli per mL and 3.0×10⁶ Photobacterium loeignathi per mL, was required for 50% survival of B. bacteriovorus. Optimal growth of B. bacteriovorus is seen at 30° C. (range: 20° C. to 45° C.) and pH of 6.8 to 7.2 (range: 5.6 to 8.6); values which are observed for the temperature of the corneal surface in horses and the pH of tears in cattle, respectively. B. bacteriovorus is unlikely to cause mammalian cell toxicity because of its outer membrane characteristics and failure to grow in eukaryotic cells in vitro.

Reports of the use of B. bacteriovorus as a biological control or therapeutic agent are limited. Fratamico (Fratamico and Whiting. J Food Prot 1995; 58:160-164) demonstrated the potential use of B. bacteriovorus as biological control for pathogenic and spoilage organisms in food. Kadouri (Kadouri and O'Toole. Appl Environ Microbiol 2005; 71:4044-4051) demonstrated that B. bacteriovorus could attack and reduce an existing E. coli biofilm in as little as 30 min of exposure. Nakaruma (Nakamura. Am J Clin Nutr 1972; 25:1441-1451) successfully treated Shigella flexneri-induced keratoconjunctivitis in rabbits with B. bacteriovorus. In a similar in vivo model, B. bacteriovorus effectively treated experimentally induced Pseudomonas aeruginosa infection in an animal model (personal communication). More recently, a group of researchers (Atterbury, Hobley, Till, et al. Appl Environ Microbiol 2011; 77(16):5794-5803) showed that young chicks experimentally infected with Salmonella enterica serovar enteritidis and dosed orally with B. bacteriovorus had a decrease in fecal shedding of Salmonella, and a reduction in cecal inflammation compared to non-treated chicks.

The present invention capitalizes on the capabilities of B. bacteriovorus and provides methods and compositions to treat and prevent infections in livestock using the bacterium. Use of the bacterium advantageously provides a highly desirable non-chemotherapeutic approach to the treatment and/or prevention of infections in animals.

Strains of B. bacteriovorus that may be utilized in the practice of the invention include but are not limited to: B. bacteriovorus strain 109J.

Types of animals that may benefit from the practice of the invention include any that are susceptible to infection by an etiological agent that can be destroyed by B. bacteriovorus. Exemplary animals include but are not limited to: members of the biological subfamily Bovinae which includes medium- to large-sized ungulates such as domestic dairy and beef cattle, bison, African buffalo, the water buffalo, the yak, and the four-horned and spiral-horned antelopes, etc. The animals may be so-called livestock raised in an agricultural setting for the production of dairy products or meat; or may be raised to perform work; or may be in another setting, e.g. in a zoo, animal reserve, etc., or raised for some other reason, e.g. as pets, show animals, for breeding purposes, etc.

Types of infections and/or diseases that may be prevented or treated by the practice of the invention are any which are caused by a bacterium that is susceptible to infection and killing by B. bacteriovorus. Exemplary diseases include but are not limited to: IBK (typically caused by Moraxella bovis, Moraxella bovoculi); BRD (e.g. caused by one or more bacteria such as Mannheimia haemolytica, Pasteurella multocida, and Histophilus somni).

In one embodiment, the animal that is treated is a bovine and the disease that is treated is IBK and/or BRD.

The invention also encompasses methods of killing bacteria such as Moraxella bovis, Moraxella bovoculi, Mannheimia haemolytica, Pasteurella multocida, Histophilus somni, especially in the context of and located at a site of bacterial infection, or a site which is susceptible to bacterial infection. The method comprises exposing the unwanted bacteria to B. bacteriovorus bacteria in an amount and under conditions sufficient for the B. bacteriovorus to infect and lyse and/or otherwise destroy (kill) the unwanted bacteria. Suitable sites of bacterial infection include but are not limited to: the eye and the area surrounding the eye and the nasopharygeal tract.

The invention also encompasses compositions, e.g. pharmaceutical compositions or preparations that may be used to carry out the methods of the invention. The compositions generally comprise B. bacteriovorus and a pharmaceutically acceptable carrier, which may be a carrier that is suitable for veterinary purposes. Such compositions are usually liquid in nature for ease of administration and are suitable for the maintenance of live B. bacteriovorus therein. The preparation of such compositions is generally known to those of skill in the art. Typically, such compositions are prepared either as liquid solutions or suspensions. Solid forms of bacterial preparations such as tablets, pills, powders (e.g. lyophilized preparations) and the like are also contemplated, especially solid forms suitable for solution in, or suspension in, liquids prior to administration. The preparation may also be emulsified or aerosolized. The bacteria may be mixed with excipients which are pharmaceutically acceptable and compatible with the bacteria. Suitable excipients are, for example, water, saline, dextrose, glycerol, ethanol and the like, or combinations thereof. In addition, the composition may contain minor amounts of auxiliary substances such as salts, wetting or emulsifying agents, pH buffering agents, and the like. The composition of the present invention may contain any such additional ingredients so as to provide the composition in a form suitable for administration. The final amount of bacteria in the formulations may vary. However, in general, the amount in the formulations will be from about 1-99%. The titer of bacteria in a preparation for administration is generally from about 1×10⁶ to about 1×10¹⁰ per mL.

The compositions of the invention may be administered by any of the many suitable means which are known to those of skill in the art. Generally, administration will be topical, including administration to any accessible surface that is infected or liable to infection by the bacteria, e.g. intraocular, intranasal, or to the skin, etc. As such, the compositions may be formulated e.g. as eye drops, sprays, mists, etc. In preferred embodiments, the mode of administration is intraocular or intranasal. In addition, the compositions may be administered in conjunction with other treatment modalities such as substances that boost the immune system, various chemotherapeutic agents, antibiotic agents, and the like.

In one embodiment, the invention encompasses an ophthalmic formulation of B. bacteriovorus (using eye drops, sprays, mists, etc.) for the treatment of cattle infected with, or suspected of being infected with, IBK. This formulation releases a therapeutic concentration of B. bacteriovorus on the eye surface for up to 24 hours or longer, depending on the levels of M. bovis present at the eye surface, since the survival of B. bacteriovorus is dependent on the presence of suitable Gram-negative bacteria as a food supply. To administer the treatment, affected animals are typically physically restrained in a chute or other suitable stanchion device.

Administration of the predatory bacterium B. bacteriovorus (e.g. via ocular instillation) provides an attractive alternative to antibiotics, especially in case of multidrug-resistant IBK infection. Its use prevents tissue and milk residues, and therefore the compositions can be used on organic farms where antibiotic administration is not allowed. Their use also decreases the opportunity for development of bacterial resistance in treated animals (and human populations consuming cattle byproducts), and avoids local and systemic side effects associated with antibiotic administration. In contrast with commercially available IBK vaccines, variation in M. bovis strains across different parts of the country is not expected to affect the killing efficiency of B. bacteriovorus.

In another embodiment, the invention encompasses a formulation of B. bacteriovorus suitable for intranasal administration for the treatment of cattle infected with or suspected of being infected with BRD, or to prevent development of BRD. Target populations of animals include but are not limited to high risk feedlot cattle. In various embodiments, the formulation releases a therapeutic concentration of B. bacteriovorus into the nasal passages and upper respiratory tract of cattle for up to 24 hours or longer, the latter depending on the levels of M. haemolytica and P. multocida present locally. The predatory bacterium B. bacteriovorus administered intranasally provides an attractive alternative to antibiotics used in metaphylaxis for prevention of BRD. Its use may prevent tissue residues, and local and systemic side effects associated with antibiotic administration. Further, due to its non-specific predatory life cycle on Gram-negative bacteria, its use would take into account the effect of variation in bacterial antigenic strains present in the upper respiratory tract of feedlot cattle. Bacterial culture of samples obtained from sick animals is the diagnostic test of choice to aid in determining the pathogens involved in BRD, and can aid in the identification of animals that can benefit from the practice of the invention. These can be obtained using nasopharyngeal swabs, transtracheal washes or bronchoalveolar lavage.

In addition to treatment purposes, the compositions and methods of the invention are also suitable for use in preventive measures. For example, in case of an outbreak of infection, administration of B. bacteriovorus to non-affected animals, especially those which are in close contact with those showing clinical signs of disease, could prevent the spread of the infection. In addition, such prophylactic treatment might be undertaken in bovines considered to be vulnerable to infection and/or in whom infection could have grave consequences, e.g. calves, show cattle, pregnant females, prize bulls, etc., whether or not an outbreak is known to have occurred.

The invention also provides methods for the long term preservation of B. bacteriovorus via lyophilization (freeze-drying). The methods involve culturing B. bacteriovorus in a suitable media under conditions sufficient for growth to achieve a density of from about 1×10⁷ to 1×10¹⁰ genomic equivalence per ml of culture medium. The culture is then concentrated using any of several methods known in the art (e.g. via centrifugation). The concentrated microorganisms are then resuspended in a suitable lyophilization medium (e.g. Microbial freeze drying buffers known in the art) and the resulting suspension is rapidly freeze-dried. Using this technique, long term storage of B. bacteriovorus (e.g. up to at least about 12 months, e.g. for 1, 2, 3, 4, 5, 6, 7, 8, 9, 10, 11 or 12 months) in a viable state is possible. The lyophilized composition thus comprises B. bacteriovorus and a suitable carrier in freeze-dried form. The viable bacteria can be revived or resurrected (reconstituted) by resuspension in a suitable media (e.g. water, saline, etc.), followed by inoculation into suitable media, usually containing a food source such as M. bovis, for further growth. Alternatively, the lyophilized bacteria may be resuspended directly, or from a water or saline suspension) into a medium suitable for administration to a subject for the treatment of an infection as described herein. For example, the B. bacteriovorus may be suspended in artificial tears or some other physiological and ocularly compatible liquid medium, e.g. Bion® tears (Alcon). Alternatively, the formulation may be made by reconstituting the lyophilized preparation in a manner that is suitable for intranasal delivery of B. bacteriovorus, e.g. in a carrier suitable for intranasal delivery, for example, a liquid which can be delivered as an aerosol, a mist, etc.

### EXAMPLES

### Example 1

Bdellovibrio bacteriovorus is a small (0.35×1.2 μm), obligate aerobe, motile (polar flagellum) gram-negative bacterium with obligate host-dependency on a wide range of Gram-negative prey bacteria. B. bacteriovorus are ubiquitous and have been isolated from terrestrial and aquatic environments including soils, rice paddies, rhizosphere of plants, rivers, sewage, fish ponds, and irrigation water. Despite the use of E. coli as the model prey bacterium in the majority of in vitro experiments published, B. bacteriovorus is selectively active against most Pseudomonas spp. and enterobacteria. Although variable between prey cells, a minimum prey density is required to sustain the B. bacteriovorus life cycle. In 2 different studies, prey concentrations of approximately 1.5×10⁵ E. coli per mL (Hespell, Thomashow, Rittenberg. Arch Microbiol 1974; 97:313-327) and 3.0×10⁶ Photobacterium leignathi per mL (Varon and Zeigler. Appl Environ Microbiol 1978; 36(1):11-17), was required for 50% survival of B. bacteriovorus. Optimal growth of B. bacteriovorus is seen at 30° C. (range: 20° C. to 45° C.) and pH of 6.8 to 7.2 (range: 5.6 to 8.6); values observed for the temperature of the corneal surface in horses and pH of tears in cattle, respectively. B. bacteriovorus is unlikely to cause mammalian cell toxicity because of its outer membrane characteristics and failure to grow in eukaryotic cells in vitro.

We have previously shown that B. bacteriovorus can reduce the number of M. bovis attached to bovine epithelial cells in an in vitro model of IBK and that B. bacteriovorus can be trained to kill M. bovis as effectively as E. coli using serial passages (Boileau, Clinkenbeard and Iandolo. Can J Vet Res 2011; 75:285-291). However, the specific/non-specific antimicrobial action of the tear film proteins, immunoglobulins (IgA) and enzymes on B. bacteriovorus viability, and hence the application of the technology in vivo, has not been investigated. An in vitro study was therefore conducted to determine whether B. bacteriovorus can remain viable in bovine tears without its prey for up to 24 hours (Boileau and Clinkenbeard. J Vet Int Med 2011; 25(3):759-760).

Tears were collected from both eyes of one clinically healthy steer by placing Wek-cell cellulose sponges in the lower medial conjunctival fornix until saturated. Tears were extracted from the sponges by centrifugation (4000 rpm×10 min), filtered twice sequentially through 0.8 μm, then 0.45 μm membranes, and frozen at −20° C. for later use.

Using a plaque assay to quantify the mean amount (±SD) of plaque forming units (PFUs) of B. bacteriovorus, the viability of active B. bacteriovorus inocula incubated in its preferred media peptone yeast extract (PYE) was compared to B. bacteriovorus viability in bovine tears or phosphate buffered saline (PBS) at time 0, 2, 4, 12, and 24 hours (Table 1-3).

Based on the PFUs of B. bacteriovorus exposed to each treatment, it was determined that viability of B. bacteriovorus over time was comparable between treatment groups. Overall, the results supported that B. bacteriovorus can remain viable in tears for up to 24 hours in the absence of prey bacteria.

TABLE 1
Comparison of B. bacteriovorus viability
in PYE and bovine tears over time

| Time | B. bdellovibrio in PYE | B. bdellovibrio in tears |
|---|---|---|
| (hr) | (×10⁹ PFU/mL) | (×10⁹ PFU/mL) |
| 0 | 5.2 ± 1.89 | 4.3 ± 1.04 |
| 2 | 4.7 ± 1.52 | 4.7 ± 1.04 |
| 4 | 3.3 ± 0.29 | 3.8 ± 1.6 |

TABLE 2
Comparison of B. bacteriovorus
viability in PYE and PBS over time

| Time | B. bdellovibrio in PYE | B. bdellovibrio in PBS |
|---|---|---|
| (hr) | (×10⁹ PFU/mL) | (×10⁹ PFU/mL) |
| 0 | 5.8 ± 4.93 | 5.6 ± 3.32 |
| 2 | 5.3 ± 2.47 | 4.2 ± 0.57 |
| 4 | 4.8 ± 1.44 | 3.3 ± 1.53 |

TABLE 3
Comparison of B. bacteriovorus viability
in PYE and bovine tears over a 24-hour period

| Time | B. bdellovibrio in PYE | B. bdellovibrio in tears |
|---|---|---|
| (hr) | (×10⁹ PFU/mL) | (×10⁹ PFU/mL) |
| 0 | 5.1 ± 0.65 | 5.1 ± 2.10 |
| 12 | 2.6 ± 1.78 | 2.7 ± 0.70 |
| 24 | 5.7. ± 0.55 | 6.3 ± 0.74 |

### Example 2

### In Vitro Investigations

Our laboratory has been actively investigating the therapeutic potential of B. bacteriovorus, a predatory bacterium capable of attacking and killing other Gram-negative bacteria including the IBK agent M. bovis, as a new treatment for IBK. Data presented in Example 1 showed that ocular instillation of B. bacteriovorus in healthy calves is safe and that it remains viable in cattle tears for up to 24 hours in the absence of prey bacteria. The present study was undertaken to investigate whether B. bacteriovorus can act as an effective M. bovis predator at prey levels present in IBK infected corneal epithelia and ocular secretions.

### Materials and Methods—Bacterial Strains.

Non-hemolytic M. bovis strain M⁻ was utilized in the investigation.

Routine cultivation of B. bacteriovorus.

Cultivation of B. bacteriovorus on E. coli was modified from that described by Ruby (Ruby. In: Balows, Truper, Dworking, Harder, Schleifer, eds. The Prokaryotes. 2^{nd} ed. New York Springer-Verlag, 1991:3400-3415). Escherichia coli was grown overnight as lawns on 5% brain heart infusion agar with 5% sheep blood (BAP) at 37° C. Bacteria were swabbed from the surface of the BAP and used to inoculate 45 mL of dilute nutrient broth (0.16% nutrient broth, 0.02% yeast extract, and 0.1% casitone supplemented with 2 mM CaCl₂ and 3 mM MgCl₂) to an absorbance reading of 0.4 to 0.6 at 600 nm. The cells were immediately harvested by centrifugation (1800×g for 5 min), the supernatant was discarded and the pellet was resuspended in 35 mL of peptone yeast extract (PYE) (1% Bacto peptone and 0.3% yeast extract supplemented with 2 mM CaCl₂ and 3 mM MgCl₂). The bacterial suspension was centrifuged as described, and resuspended in 5 mL of PYE to a final concentration of approximately 1×10⁹ E. coli/mL. A 75 cm² flask (Cell star tissue culture flask; Greiner Bio-One, Frickenhansen, Germany) containing 20 mL of PYE was inoculated with 5 mL of E. coli and 250 μl of an active 7 to 10 d old stock culture of B. bacteriovorus [Multiplicity of Infection (MOI)=1; ratio 1 E. coli:1 B. bacteriovorus], and incubated with shaking (180 rpm) at 30° C. After approximately 48 h of incubation, the solution was examined microscopically (Olympus BX41 laboratory microscope; Hitschfel Instruments, St-Louis, Mo., USA) and considered active and ready to use when it contained motile, active attack-phase B. bacteriovorus (1×10⁹ plaque forming units (PFU)/mL) with no visible E. coli or bdelloplasts (infected E. coli cells). Once active, B. bacteriovorus culture was stored at 4° C. for a maximum period of 1 mo.

Cultivation of B. bacteriovorus Using M. bovis as Prey.

Moraxella bovis prey cells were prepared exactly as the E. coli inoculum. Motile and active B. bacteriovorus previously grown on E. coli were harvested as described by Rogosky (Rogosky, Moal and Emmert. Current Microbiol 2006; 52:81-85). The E. coli cell debris was removed by centrifugation (1500×g for 5 min). The B. bacteriovorus-rich supernatant was saved and the cells were centrifuged (8500×g for 20 min), washed in PYE, centrifuged again (8500×g for 20 min), and resuspended in PYE. The washed cells were filtered 3 times sequentially through 0.8 μm, then 0.45 μm, and finally 0.45 μm membranes (Acrodisc syringe filters; Pall Corporation, Ann Arbor, Mich., USA) pre-wetted with PYE to remove residual E. coli cell debris. A 75 cm² flask containing 15 mL of PYE was inoculated with 5 mL of M. bovis and 5 mL of triple filtered B. bacteriovorus [MOI=0.2; ratio 1 M. bovis:5 B. bdellovibrio], and incubated with shaking (180 rpm) at 30° C. for a period of 5 to 7 d or until active. Once grown on M. bovis, subsequent subcultures were done using the same protocol, without filtration.

Preparation of M. bovis Inoculum.

Lawns of M. bovis were grown for 24 h at 37° C. on BAP. Cells were harvested by centrifugation (13 000×g for 15 min), washed then diluted in Hank's balanced salt solution containing phenol red and supplemented with 25 μg/mL of glucose (HBSS+G) to an absorbance reading of 0.14 at 600 nm, for a final bacterial concentration of 4×10′ M. bovis/mL.

Preparation of B. bacteriovorus Inoculum.

Ten milliliters of active and motile B. bacteriovorus previously grown on M. bovis was centrifuged (1500×g for 5 min) to remove cell debris. The B. bacteriovorus-rich supernatant was saved and the cells were centrifuged (8500×g for 20 min) and resuspended in PYE. The washed B. bacteriovorus cells were filtered twice (0.8 μm, 0.45 μm) to remove residual M. bovis cell debris. This suspension was cultured on BAP and was shown to be free of M. bovis. However, this filtration process also decreased the number of B. bacteriovorus present by 30% or greater. Therefore, the number of active B. bacteriovorus in the filtered bacterial suspension was estimated under light microscopy (100×).

Enumeration of B. bacteriovorus Using Plaque Assays.

A modification of the double-layer plaque assay technique used for counting bacteriophage described by Varon and Shilo (Varon and Shilo. J Bacteriol 1968:744-753) was used for enumeration of B. bacteriovorus. A 200 μL sample of B. bacteriovorus dilutions 10⁻⁵, 10⁻⁶, 10⁻⁷, 10⁻⁸, and 10⁻⁹ and 200 μL of the E. coli suspension (as prepared in routine cultivation of B. bacteriovorus) were mixed in 3 mL of liquefied overlay agar (PYE medium containing 0.6% agar) kept on a hot plate at 45° C. The mixtures were inverted 6 or 7 times to allow proper mixing and immediately spread over the surface of PYE medium containing 1.5% agar in 92×16 mm in Petri dishes. Plates were incubated upright at 30° C. for 3 to 7 d until clear circular plaques appeared in the lawn of prey cells.

### Efficiency of Killing Assay.

Bdellovibrio bacteriovorus and M. bovis inocula were prepared as described except that prey cells were resuspended in HBSS to a final M. bovis concentration of approximately 1×10⁸/mL. The number of live B. bacteriovorus present in the inoculum was determined via plaque assay technique. Five hundred microliters of the original M. bovis inoculum was serially diluted in 4.5 mL of HBSS (10⁻¹ to 10⁻⁴). In group A (control group), 50 μL of PYE was added to triplicate tubes of 250 μL of each M. bovis dilutions (1×10⁴/mL to 1×10⁸/mL). The same method was used for group B (treatment group), except that PYE was replaced by 50 μL of B. bacteriovorus inoculum (1×10¹⁰ PFU/mL), resulting in a final 1:1 predator to prey ratio. Serial dilution tubes were incubated at 35° C. for 4 h with shaking (180 rpm). To determine the M. bovis colony forming units (CFU)/mL at time 0 and 4 h, each M. bovis dilution from the 3 replicates of group A and B were further serially diluted, and serial dilution 10⁻¹, 10⁻², 10⁻³, and 10⁻⁴ were plated on BAP and incubated at 37° C. for 24 h. The percent of M. bovis killed by B. Bdellovibrio predation was calculated using the formula described previously by Rogosky (17):

$$\% killed = ( 1 - \underline{{B}_{4} / {B}_{0}} ) \times 100$$

$${A}_{4} / {A}_{0}$$

where: A₀ and A₄ are the mean CFU/mL M. bovis in the absence of B. bacteriovorus (group A) at 0 and 4 h, respectively, and B₀ and B₄ are the mean CFU/mL M. bovis in the presence of B. bacteriovorus (group B) at 0 and 4 h.

### Madin-Darby Bovine Kidney Cell Culture.

Immortalized MDBK cells (50th to 60th passage levels) were utilized in this investigation. Tissue culture growth media consisted of Dulbecco's modification of Eagle's medium with glucose (4.5 g/L) and L-glutamine (DMEM), supplemented with 10% (v/v) fetal bovine serum (FBS) and 1% (v/v) of penicillin-streptomycin (10 000 IU/mL, 10 000 μg/mL). For the adherence assay, antibiotic-free media was used. Cells were incubated at 37° C., 5% CO₂ and 90% to 100% humidity and cell culture media was changed 3 times weekly until cells reached 90% confluency. Cells formed monolayers within 3 to 4 d of incubation, and additional passages of the cells were done using conventional procedures at a split ratio of 1:14.

### Cell Monolayers on Coverslips.

Madin-Darby bovine kidney cells were grown as monolayers on coverslips for adherence experiments as described by Moore and Rutter (Moore and Rutter. Aust Vet J 1989; 66:39-42). Prior to cell seeding, 13 mm round coverslips (Thermanox cell culture coverslips; Nalge Nunc International, Rochester, N.Y., USA) were placed at the bottom of each well of four 24-well tissue culture plates coated with type 1 collagen (Greiner Bio-One, Frichenhansen, Germany). The cell culture was maintained coated side up. Ninety percent confluent MDBK monolayers were released from their original tissue culture flask with trypsin-EDTA (0.05%, 0.53 mM EDTA). The detached cells were centrifuged (200×g for 5 min) and then resuspended in fresh tissue culture growth media. The suspension was adjusted to 2×10⁵ viable cells per mL. Four 24-well plates were seeded with 1×10⁵ cells per coverslip. Each coverslip was incubated in their respective well with 0.5 mL of cell suspension at 37° C., 5% CO₂, and 90% to 100% humidity. The cells were used after 36 to 48 h when the monolayers were 75% to 80% confluent as estimated by light microscopy (Olympus CKX-41 inverted microscope; Hitschfel Instruments, St-Louis, Mo., USA).

### Attachment Assay.

Attachment of M. bovis to MDBK cells was done as described by Annuar and Wilcox (Annuar and Wilcox. Res Vet Sci 1985; 39:241-246), with slight modifications. Bdellovibrio bacteriovorus and M. bovis inocula, as well as MDBK monolayers grown on coverslips, were prepared as described. The media type and amount per well, incubation temperature and time, and the concentration of the bacterial suspension used were determined by preliminary experiments (data not shown) to give optimum MDBK cells viability, preserve M. bovis viability and prevent overgrowth, provide optimum M. bovis attachment to MDBK cells, and mimic what would occur at the level of the ocular surface in case of naturally occurring IBK.

To begin the assay, tissue culture growth media was removed from each well of 24-well plate group 1 (control plate) and group 3 (B. bacteriovorus plate), and replaced by 400 μL of HBSS+G. The same process was repeated for group 2 (M. bovis plate) and group 4 (M. bovis+B. bacteriovorus plate) except that each well received 400 μL of M. bovis inoculum. All 4 plates were incubated at 37° C. for 45 min. Following incubation, coverslips were transferred to 4 new corresponding 24-well tissue culture plates using 25 gauge needles and sterile tissue forceps with non-serrated tips leaving the majority of the non-adherent M. bovis inocula in the original wells. The coverslips were not washed to remove residual bacteria inocula because this process also resulted in detachment of variable numbers of MDBK cells. At time zero, 250 μL of HBSS+G and 50 μL of PYE was added to each well of plate 1 and plate 2, and 250 μL of HBSS+G and 50 μL of B. bacteriovorus inoculum was added to each well of plate 3 and plate 4. To determine the CFUs per well at 0, 6, and 12 h, media from 6 randomly selected wells from each treatment group was removed, serially diluted (up to 10⁻³), and plated on BAP, which were incubated at 37° C. for 24 h. The MDBK coverslips from corresponding wells were fixed in methanol and acetone (1:1 ratio) for 2 min and stained with Wright-Giemsa stain with phosphate buffered saline added (3:1 ratio) for 10 min. To quantify the mean number of adherent bacteria per MDBK cell, attached M. bovis were counted on 40 MDBK cells per coverslip. The number of live B. bacteriovorus present in the inoculum in the combined media from 2 randomly selected wells in plate 3 (B. bacteriovorus group) and plate 4 (M. bovis+B. bacteriovorus group) at time 0 and at 12 h, was determined via plaque assay technique.

Efficiency of B. bacteriovorus Predation Following Serial Passages on M. bovis.

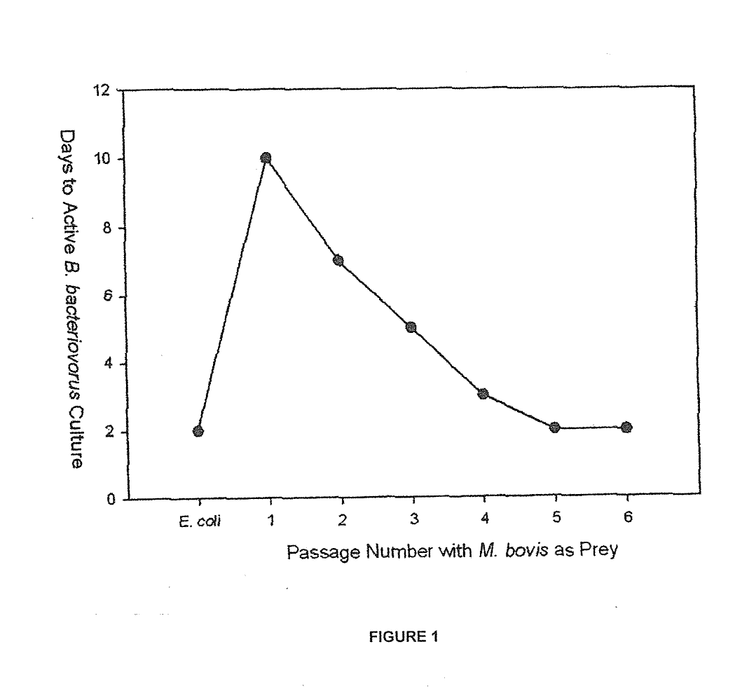

As shown in FIG. 1, initial passage of B. bacteriovorus using M. bovis as prey required 10 days for active cultures to develop compared with 2 days for culture on normal E. coli prey; however by the 5th passage of B. bacteriovorus on M. bovis, time to active predatory morphology was reduced to 2 days.

Efficacy of B. bacteriovorus Predation of M. bovis in Broth Cultures.

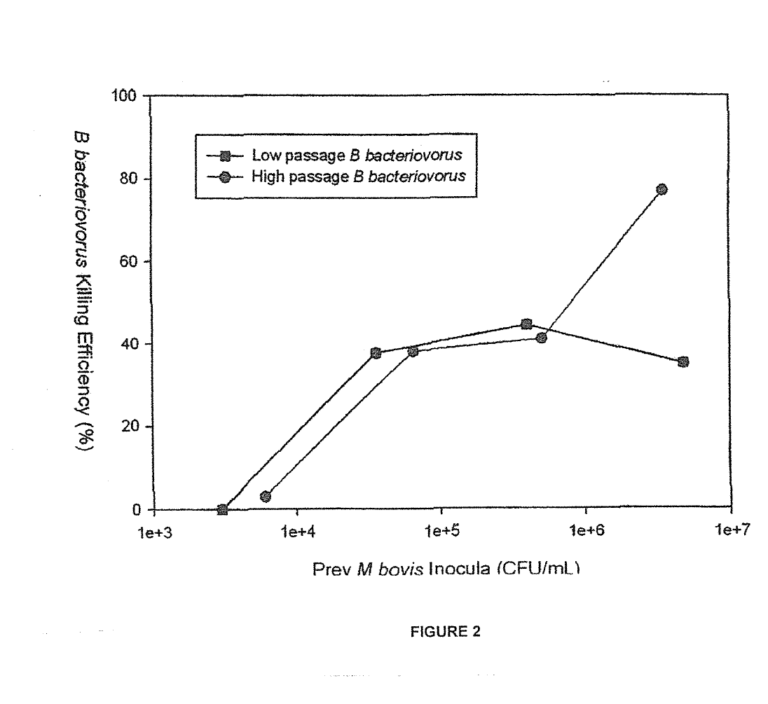

The level of M. bovis prey required for B. bacteriovorus to demonstrate predation was assessed in suspension broth cultures for M. bovis prey levels of 1×10³ to 1×10⁷ CFU/mL with a B. bacteriovorus inoculum of 1×10¹⁰ PFU/mL. As shown in FIG. 2, B. bacteriovorus passaged on M. bovis 3 times showed killing of ˜40% for M. bovis prey levels greater than 4×10⁴ CFU/mL. However after 6 passages, killing efficiency increased to ˜76% only for M. bovis prey levels greater than 9×10⁶ CFU/mL.

Efficacy of B. bacteriovorus Predation of M. bovis in an In Vitro Model of IBK.

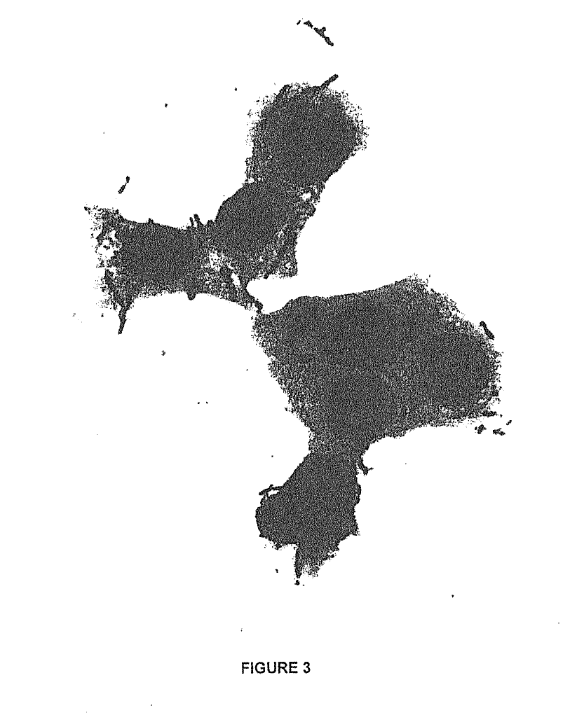

Moraxella bovis attaches specifically to epithelial cells of bovine origin (Kangonyera, George and Munn. Am J Vet Res 1989; 50:10-17; Jackman and Rosenbush. Curr Eye Res 1984; 3:1107-1112; Moore and Rutter. Aust Vet J 1989; 66:39-42.), and MDBK cells have been used by others as an in vitro model of M. bovis attachment for IBK (Annuar and Wilcox. Res Vet Sci 1985; 39:241-246.). Using this model M. bovis attached to MDBK cells appeared as darkly stained rods or coccobacillary shapes, characteristically in pairs, adhered to the surface of the larger polygonal MDBK cells (FIG. 3). At the time of inoculation in the presence of 1.6×10¹¹ PFU/mL of B. bacteriovorus, the mean number of bacteria attached per MDBK cell was ˜4 M. bovis per MDBK cell (Table 1). The number of M. bovis attached per MDBK cell remained relatively constant during the 12 hours incubation period with no statistical difference between the 0, 6, and 12 hours time points for the controls lacking B. bacteriovorus. When compared with the presence of B. bacteriovorus, there was 1.9-fold decrease of M. bovis attached to MDBK cells after 6 hours and 6-fold decrease after 12 hours, which were statistically significant at P<0.05 and P<0.001, respectively.

TABLE I
Bdellovibrio bacteriouvorus clearance of Moraxella bovis
attached to Madin-Darby bovine kidney (MDBK) cells.

| | Attachment | | Unattached | |
|---|---|---|---|---|
| | (M. bovis per MDBK)^{e} | | M. bovis (10³ CFU/mL) | |
| Exposure | No | With | No | With |
| time | B. bacteri- | B. bacteri- | B. bacteri- | B. bacteri- |
| (h) | ovorus | ovorus | ovorus | ovorus |
| 0 | 4.13 ± 2.62 | 4.62 ± 1.31 | 80 ± 152^{c} | 998 ± 571^{c} |
| 6 | 3.41 ± 1.27^{a} | 1.77 ± 0.45^{a} | 277 ± 65 | 213 ± 87 |
| 12 | 5.35 ± 1.96^{b} | 0.88 ± 0.11^{b} | 1.520 ± 860^{d} | 691 ± 72 |

^{a}Statistically significant at P < 0.05
^{b}Statistically significant at P < 0.001
^{c}Statistically significant at P < 0.005
^{d}Statistically significant at P < 0.005
^{e}Results represent mean ± 1 standard deviation from 6 coverslips.

In addition to M. bovis attached to MDBK cells, 0.8×10⁵ to 1×10⁶ CFU/mL M. bovis were present in the culture media at the beginning of the incubation period (Table I). In the absence of B. bacteriovorus, the M. bovis level increased to 1.5×10⁶ CFU/mL at 12 hours, whereas in the presence of B. bacteriovorus the supernatant M. bovis declined at 6 hours and were near the pre-inoculation value of 0.7×10⁶ CFU/mL at 12 hours. These values were significantly different (P<0.005) from that for the supernatant M. bovis CFU/mL in the absence of B. bacteriovorus.

The supernatant B. bacteriovorus level increased from 8×10⁹ PFU/mL at 0 h to 3×10¹⁰ PFU/mL and 5×10¹⁰ PFU/mL at 12 hours in the absence and presence of M. bovis, respectively. The number of active B. bacteriovorus in the original filtered bacterial suspension ranged from 15 to 20 per 100× field under light microscopy.

Efficacy of Low and High Passages B. bacteriovorus Predation of M. bovis in Suspension Broth Cultures.

It is difficult to replicate the in vivo conditions of IBK in an in vitro model for assessing the efficiency of predation of M. bovis by B. bacteriovorus. The 4 hours time period was selected to provide sufficient time for B. bacteriovorus to complete its life cycle and the incubation temperature of 35° C. was selected (instead of the 30° C. optimum for B. bacteriovorus) for optimal M. bovis growth and viability. Bdellovibrio bacteriovorus requires adequate levels of prey bacteria, typically >1×10⁶ CFU/mL, in order to maintain its predatory lifestyle. The efficiency of predation followed a predicable trend in that at low prey levels (<10⁴ CFU/mL) there were insufficient prey to either trigger or sustain B. bacteriovorus predation, but at higher prey levels (>10⁵ CFU/mL and >10′ CFU/mL) efficiency of predation increased from ˜40% to ˜76%, respectively. The range of this amount is less efficient compared with the reported B. bacteriovorus predation efficiency of other gram-negative bacteria such as E. coli, but comparable to various strains of Enterobacter spp, Erwinia spp, and Salmonella spp. Rogosky et al (Rogoski, Moak, Emmert. Current Microbiol 2006; 52:81-85) reported that B. bacteriovorus does have prey preference, killing some prey more efficiently than others. Pantoea agglomerans, E. coli, and S. marcescens were reported to be preferred prey for B. bacteriovorus with efficiency of predation of ˜90% compared with E. aerogenens, E. carotova subsp. carotova, and S. enterica with efficiency of predation of ˜60%. It has been observed here and by others that B. bacteriovorus can be trained to kill less preferred prey more efficiently by continuous passage on that prey. For this study, B. bacteriovorus was initially passaged on M. bovis 3 times with modest shortening of the passage time (from 10 days to 5 days after 3rd passage) to fully active B. bacteriovorus cultures. Improved efficiency of predation at high prey concentration (>10⁷ CFU/mL) was attained when B. bacteriovorus number of passages on M. bovis increased to 5 times, with corresponding passage time of 2 days to fully active B. bacteriovorus culture (FIG. 1).

Efficacy of B. bacteriovorus predation of M. bovis in an in vitro model of IBK.

Our initial experiments with the in vitro IBK model used a β-hemolytic strain of M. bovis, but this strain produced significant cytotoxicity resulting in MDBK cell detachment from coverslips such that the mean number of M. bovis attached per MDBK cell could not be consistently determined. Therefore, a non β-hemolytic strain of M. bovis was used in this study. No difference in efficiency of predation by B. bacteriovorus of hemolytic versus non-hemolytic strains of M. bovis was observed.

A paramount question for the use of B. bacteriovorus as a non-chemotherapeutic alternative therapy for IBK is whether B. bacteriovorus can efficiently decrease the number of M. bovis adhered to corneal epithelial cells in infected bovine eyes. To study this in vitro, a co-culture model of M. bovis with MDBK cells was used, with the media selected to support both MDBK and M. bovis viability while attempting to mimic the ocular environment. To reproduce the physical ocular environment, round coverslips with the approximate dimension of the bovine cornea were used and to mimic the ocular tear film, the media column above the coverslip was kept to the minimum that supported MDBK cell viability for a 12 hours exposure period. Selected media was also tailored to simulate tears. It contained minimum levels of nutrients in an aqueous salts composition to reduce M. bovis growth rate to one simulating the slower growth rate in vivo. This was accomplished in that the doubling time for M. bovis in the in vitro IBK model was 4.3 hours (calculated from data for Table I). During the 12 hours experimental period, M. bovis in the unexposed control IBK model underwent approximately 3 doublings, but M. bovis attachment to MDBK cells only increased 1.3-fold. However, exposure to B. bacteriovorus decreased the number of adherent M. bovis on MDBK cells in vitro by 6-fold (Table I).

B. bacteriovorus appears to be an effective predator of bacterial prey fixed on surfaces as demonstrated by its efficacy against bacteria in surface biofilms (Kadouri and O'Toole. Appl Environ Microbiol 2005; 71:4044-4051). Exposure to B. bacteriovorus increased the levels of planktonic M. bovis by 12-fold at the beginning of the experiment, suggesting that the predatory bacteria is efficient in preventing initial attachment of M. bovis on MDBK cells in vitro. Although B. bacteriovorus was not as effective in clearing M. bovis from the aqueous media above the coverslip, it did reduce the CFU/mL by 1.4-fold as compared to a 19-fold increase in the controls without B. bacteriovorus (Table I). The apparent bactericidal effect of M. bovis attached to bovine epithelial cells may be crucial for reducing M. bovis ulceration of the cornea of infected cattle because pathogenesis of these lesions appears to be the result of direct contact of M. bovis with corneal epithelial cells. In contrast, although M. bovis attached to corneal epithelia extend into the corneal stroma where they may be protected from innate and acquired immunity, M. bovis in the tear film will not only be exposed to B. bacteriovorus predation but also to innate immune response of infiltrating neutrophils; tear antimicrobial proteins and enzymes, such as lysozyme, the latter reported to increase up to 33-fold in cattle with inflamed corneas; and to acquired immunity through secreted IgA and leaked serum IgG, and therefore, bacteriostatic activity of B. bacteriovorus against M. bovis in the tear film may be adequate to resolve infection. Due to its poor specificity against gram-negative bacteria, the true protective effect of tear lysozyme against the invasion of the ocular surfaces by M. bovis is unknown.

In summary, the present study confirms that B. bacteriovorus can be trained to kill M. bovis as effectively as its normal prey E. coli. The efficiency of low passage B. bacteriovorus predation on M. bovis in suspension broth culture was ˜40% at prey levels >4×10⁴ CFU/mL though, at high passage on M. bovis and prey levels >9×10⁶ CFU/mL, the efficiency was increased to ˜76%. The minimum M. bovis concentration to sustain B. bacteriovorus life cycle was established to be <10⁴ CFU/mL. In the in vitro model of IBK, exposure to B. bacteriovorus not only significantly decreased the number of adherent M. bovis on MDBK cells, but also had a bacteriostatic effect on planktonic M. bovis. Therefore, we conclude that B. bacteriovorus can act as an effective M. bovis predator at levels present in IBK infected corneal epithelia and ocular secretions.

### Example 3

### Treatment of BRD

Bovine respiratory disease (BRD) is the most costly beef cattle disease in North America. Predisposing factors include immunocompromised host due to stress and/or viral infection that decreases innate and adaptive immune mechanisms of the respiratory tract. Current preventative measures rely on stress-minimization, the practice of metaphylaxis, and vaccination; however vaccination is not 100% effective and the practice of metaphylaxis is associated with antibiotic residue. As a result, our laboratory has investigated the potential of B. bacteriovorus, a predatory bacterium capable of inducing lysis of Gram-negative bacteria, as a non-chemotherapeutic preventative measure for BRD. The investigation involved assessing the ability of B. bacteriovorus to infest and kill the bacterium Mannheimia haemolytica, a major causative agent of BRD.

### Materials and Methods

Motile and active B. bacteriovorus previously grown on 23^{rd} pass non-hemolytic M. bovis were harvested. The non-hemolytic M. bovis cell debris was removed by centrifugation (1,250 g×8 min). The B. bacteriovorus-rich supernatant was saved, unfiltered, and the cells were centrifuged (12,000 g×20 min) then resuspended in 5 mL of peptone yeast extract (PYE). A 75 cm², non-air tight flask containing 12.5 mL of brain heart infusion (BHI) and 2.5 mL PYE was inoculated with 5 mL of M. haemolytica and 5 mL B. bacteriovorus as prepared above (ratio 1 M. haemolytica:5 B. bacteriovorus, MOI=0.2). Leukotoxin-producing M. haemolytica isolates “Okie” (Ok) and “Wild-Type” (WT), were utilized.

The flask was incubated with shaking (180 rpm) at 32° C. for the first 2 days then at 30° C. for period of 3 to 5 days or until active. Once grown on M. haemolytica, subsequent subcultures were performed weekly using the same protocol. Wet mounts under light microscopy (100×) were used to determine time to active B. bacteriovorus predatory morphology between passages on M. haemolytica.

The results showed that the average killing percentage of B. bacteriovorus on Ok-M. haemolytica and WT-M. haemolytica was 21% and 27%, respectively for all prey concentrations, supporting a conclusion that B. bacteriovorus can be trained to kill both WT-M. haemolytica and Ok-M. haemolytica.

### Example 4

### Safety of Administration

Administration of B. bacteriovorus strain 109J to the eye surface of healthy calves (n=6) was carried out and the effects were observed for up to 7 days following administration. No local and/or systemic adverse effects were observed. This outcome supports the conclusion that administration of B. bacteriovorus directly to the eye surface of bovines is safe and does not cause deleterious side effects.

### Example 5

### In Vivo Testing

Clinical trials are conducted using healthy calves that are experimentally infected with Moraxella bovis (M. bovis) respectively, are conducted to assess the potential therapeutic effect of B. bacteriovorus in the treatment of pinkeye also known as infectious bovine keratoconjunctivitis (IBK). Clinical trials can also be conducted in cattle herds with naturally occurring pinkeye. Briefly, IBK infection rate (percentage of infected eyes in which M. bovis is isolated from an eye swab on 2 or more consecutive days) and prevalence of clinical signs of IBK (percentage of infected eyes in which a corneal opacity or ulcer developed) is determined. Infected calves are treated with B. bacteriovorus as described herein (e.g. administered at least once by ocular instillation). Statistical analysis of the data shows that administration of B. bacteriovorus lessens M. bovis levels in tears and/or abolishes clinical signs of IBK in the infected eyes.

### Example 6

### B. bacteriovorus Lyophilization (Freeze-Drying) Technique

We have developed a stable B. bacteriovorus product using lyophilization. Briefly, motile and active B. bacteriovorus previously grown on non-hemolytic M. bovis were harvested via centrifugation (10,000 g×20 min). The supernatant was discarded and the B. bacteriovorus “pellet” was reconstituted in Microbial freeze drying buffer (OPS Diagnostics, LLC).

Small glass freezing vials were inoculated with 1000 μL (1 mL) aliquots of this mixture which was “shell frozen” in liquid nitrogen then placed in a lyophilizing chamber which was quickly attached to a manifold under vacuum for a period of 24 hours. Once lyophilized, the vials were stored at 4° C. and protected from light.

Surprisingly, once resurrected B. bacteriovorus inoculum concentrations ranged from 1×10⁷ to 1×10¹⁰ genomic equivalence per ml (GE/mL) which is comparable to values observed prior to lyophilization. The process of resurrection includes adding 1 mL of sterile water to the lyophilized B. bacteriovorus powder to create a suspension. When inoculated with M. bovis in standard liquid culture, B. bacteriovorus becomes fully active (4+ using a conventional B. bacteriovorus activity scale) within 24 to 72 hours. The product appears to be stable over time with either 0 to <1.6 log decrease in B. bacteriovorus concentration. Current shelf-life of the product under conditions stated above is 12 months.

When preparing B. bacteriovorus inoculum for ophthalmic (topical) instillation onto cattle eyes, the lyophilized powder is reconstituted with Bion® tears (Alcon), a 0.1% Dextran+0.3% Hypomellose based eye drops.

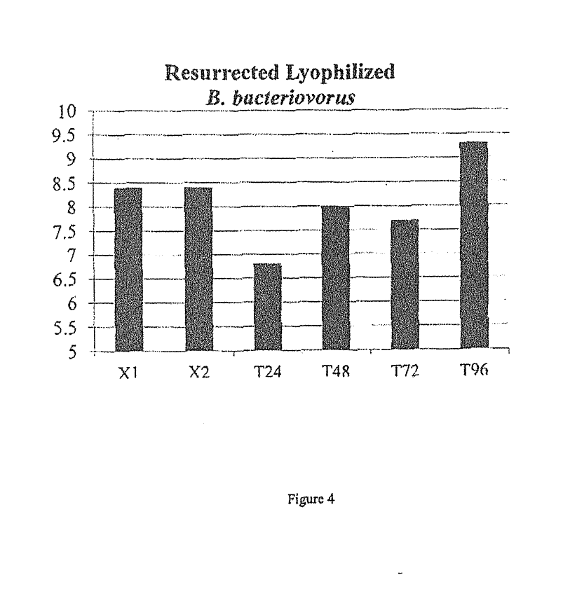

The results obtained when a resurrected B. bacteriovorus culture was grown on non-hemolytic M. bovis after 3 months of being lyophilized are shown in FIG. 4. As can be seen, bacterial losses following lyophilization ranged from only ˜0 to 1.6 log. Thus, B. bacteriovorus can be successfully preserved in a viable form in this manner.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

1. A method for treating or preventing an eye disorder in a subject in need thereof, comprising administering to said subject a therapeutic composition comprising Bdellovibrio bacteriovorus, wherein said eye disorder is infectious bovine keratoconjunctivitis (IBK).

2. (canceled)

3. The method of claim 1, wherein said therapeutic composition is administered topically to the eye.

4. The method of claim 3, wherein said therapeutic composition is administered via ocular instillation.

5. The method of claim 1, wherein said subject is a bovine.

6. A method for treating or preventing a respiratory disorder in a subject in need thereof, comprising administering to said subject a therapeutic composition comprising Bdellovibrio bacteriovorus.

7. The method of claim 6, wherein said respiratory disorder is bovine respiratory disease (BRD).

8. The method of claim 6, wherein said therapeutic composition is administered to the nasal cavity of said subject.

9. The method of claim 6, wherein said subject is a bovine.

10. A method of killing a Gram-negative bacterium, comprising the step of
exposing said Gram-negative bacterium to Bdellovibrio bacteriovorus under conditions which allow said B. bacteriovorus to infect and kill said Gram-negative bacterium,
wherein said Gram-negative bacterium is selected from the group consisting of Moraxella bovis, Moraxella bovoculi, Mannheimia haemolytica, Pasteurella multocida, and Histophilus somni).

11. A lyophilized preparation of Bdellovibrio bacteriovorus.

12. A formulation for ophthalmic delivery of Bdellovibrio bacteriovorus comprising the lyophilized preparation of claim 11 reconstituted in a carrier suitable for ophthalmic delivery.

13. The formulation of claim 12 wherein said formulation is a liquid suitable for ocular instillation.

14. A formulation for intranasal delivery of Bdellovibrio bacteriovorus comprising the lyophilized preparation of claim 11 reconstituted in a carrier suitable for intranasal delivery.

15. The formulation of claim 14, wherein said formulation is suitable for delivery as a powdered aerosol.
